(19) United States
(12) Patent Application Publication
(10) Pub. No.: US 2012/0201915 A1
(43) Pub. Date: Aug. 9, 2012

(54) THERAPEUTIC COMPOSITION

(76) Inventor: Deborah Stone, Harpersville, AL (US)

(21) Appl. No.: 13/503,321

(22) PCT Filed: Oct. 20, 2010
(86) PCT No.: PCT/US10/53427

§ 371 Date: Apr. 20, 2012

Related U.S. Application Data
(60) Provisional application No. 61/253,405, filed on Oct. 20, 2009.

(51) Int. Cl. A61K 36/185; A61K 31/23; A61P 17/02

(52) U.S. Cl. 424/776; 424/725; 514/546

(57) ABSTRACT

This application discloses a therapeutic composition, a method of preparing a therapeutic composition, and a method for treating skin with a therapeutic composition.

### 1. TECHNICAL FIELD

This disclosure relates generally to skin treatments and therapeutic compositions. More particularly, this disclosure relates to an improved healing and therapeutic composition dispensable in cream, gel or paste, or other form.

### 2. BACKGROUND

Skin is the most frequently injured of the body's organs. As the skin is in constant contact with the external environment, this organ is constantly exposed to potential injuries. Injuries to skin can result from many different events and diseases. Such damage may impair or entirely destroy the function of the injured organ or tissue, and the outcome of such damage depends on the nature and role of the tissue or organ affected. The wound healing response is most commonly described with reference to the healing of skin wounds. As wounds are painful, even aside from the events associated with their formation, delays in the healing of wounds may be associated with extended incidences of pain to the patient or animal.

Accordingly, there is always a need for improved techniques for treating skin damage or wounds. There also is a need for an improved composition for treating skin and skin wounds. It is to this need, among others, that this disclosure is directed.

### SUMMARY

This application discloses therapeutic compositions, methods for preparing therapeutic compositions, and methods for treating various skin wounds, diseases and ailments with therapeutic compositions.

### BRIEF DESCRIPTION OF THE FIGURES

FIG. 1A shows the UPLC chromatogram of an exemplary extract of Plantago in jojoba oil at the wavelength of 210 nm.

FIG. 1B shows the UPLC chromatogram of an exemplary extract of Plantago in jojoba oil at the wavelength of 254 nm.

FIG. 1C shows the UPLC chromatogram of an exemplary extract of Plantago in jojoba oil at the wavelength of 280 nm.

FIG. 1D shows the UPLC chromatogram of an exemplary extract of Plantago in jojoba oil at the wavelength of 330 nm.

FIG. 1E shows the UPLC chromatogram of an exemplary extract of Plantago in jojoba oil at the wavelength of 450 nm.

FIG. 2 shows various mass (ESI-positive ion mode) spectra of the different peaks of an exemplary extract of Plantago in jojoba oil.

### DEFINITIONS

In this specification, various terms are defined as follows. Unless otherwise defined, all technical and scientific terms used herein have the same meaning as commonly understood by one of ordinary skill in the art to which this disclosure pertains.

The compositions and methods for their use can “comprise,” “consist essentially of,” or “consist of” any of the ingredients disclosed throughout the specification.

It is contemplated that any embodiment discussed in this specification can be implemented with respect to any method or composition of the disclosure, and vice versa. Furthermore, compositions can be used to achieve methods disclosed herein

“Topical application” means to apply or spread a composition onto the surface of keratinous tissue. “Topical skin composition” includes compositions suitable for topical application on keratinous tissue. Such compositions are typically dermatologically-acceptable in that they do not have undue toxicity, incompatibility, instability, allergic response, and the like, when applied to skin. Topical skin care compositions can have a selected viscosity to avoid significant dripping or pooling after application to skin.

“Dermatologically acceptable carrier, vehicle, or medium” means a carrier, vehicle, or medium into which the active ingredients can be effectively incorporated thereinto. A dermatologically acceptable carrier can be designed to reduce or avoid undue toxicity, incompatibility, instability, allergic response, and the like, when applied to skin.

A “non-volatile oil” includes those substance that will not significantly evaporate at ordinary or room temperature.

“Nontoxic” generally refers to substances that, upon ingestion, inhalation, or absorption through the skin by a human or animal, do not cause, either acutely or chronically, damage to living tissue, impairment of the central nervous system, severe illness, or death. The term “nontoxic” also can refer to compounds or metabolites of which can be incorporated innocuously and without harm to the surrounding environment. The opposite of “nontoxic” is defined as “toxic.”

The terms “mixture,” “mix,” and “mixing” or any variants of these terms, when used in the claims and/or specification includes, stirring, blending, dispersing, milling, homogenizing, and other similar methods. The mixing of the components or ingredients of the disclosed compositions can form into a solution. In other embodiments, the mixtures may not form a solution. The ingredients/components can also exist as undissolved colloidal suspensions.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one specific embodiment substantially refers to ranges within 10%, within 5%, within 1%, or within 0.5%.

The terms “treating,” “inhibiting,” or “reducing” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the term “or” in the claims is used to mean “and/or” unless explicitly indicated to refer to alternatives only or the alternatives are mutually exclusive, although the disclosure supports a definition that refers to only alternatives and “and/or.”

The term “skin” includes the epidermis, dermis, stratum corneum or combinations thereof on a mammal, e.g. a human, horse, etc.

The term “wound(s)” can be primarily described with reference to skin wounds, which can arise from an array of circumstances including radiation treatment, injury, illness, and disease. One of ordinary skill in the art can appreciate that the acceleration of healing of wounds in accordance with this disclosure should not be limited to superficial skin wounds.

### DETAILED DESCRIPTION

Specific embodiments include therapeutic compositions, methods for preparing therapeutic compositions, and methods for treating skin or wounds with therapeutic compositions. In one embodiment, the composition comprises at least one ingredient extracted into a carrier oil (e.g. jojoba esters or jojoba oil) from a botanical material selected from the group consisting of Plantago lanceolata, Plantago major, and a combination thereof. The inventor believes that the topical application of the compositions can assist in the healing or accelerating the rate at which wounds are healed.

In one specific embodiment, a therapeutic composition comprises (1) carrier oil (e.g. jojoba oil) or Jojoba esters, (1) botanical extracts from botanical materials (e.g., Plantago Lanceolata, also called English plantain) into the carrier oil or Jojoba esters, and (3) a dermatologically acceptable carrier. This therapeutic composition further can comprise an essential oil (e.g. French lavender), and can be preserved with preservatives (e.g. vitamin E oil). Up to about 1.5% of other therapeutic essential oils also can be added to the composition to tailor the composition to specific needs or results.

The botanical materials and oils for use with specific embodiments can be harvested from traditional sources. In some examples, the Plantago lanceolata (also called English plantain) material can be harvested using ordinary techniques, and the lanceolate (spreading or erect), basal leaves can be used with this embodiment. Further, Plantago major is a species of Plantago, family Plantaginaceae and can be harvested using ordinary techniques. All of botanicals can be organic if desired. Botanical materials can be extracted into the carrier oil, jojoba esters or the silicone fluid to achieve a desired result.

Jojoba esters or oil, suitable with various embodiments, can be a hydrogenation or interesterification product of Jojoba oil. In fact, the jojoba oil can be a complex mixture of naturally occurring long-chained linear esters with many functional properties. In one embodiment, over 97% of jojoba oil was composed of an array of liquid wax esters, with a combination of mixed tocopherols, free sterols and other unsaponifiable material making up the balance. Jojoba esters may be waxes, and there may be no triglyceride component of jojoba esters. Chemically, jojoba esters can be a complex mixture of long chain (C36-C46) fatty acids and fatty alcohols joined by an ester bond. Jojoba esters can be produced by the interesterification of jojoba oil, hydrogenated jojoba oil, or a mixture of the two. In one specific embodiment, the jojoba esters does not contain any trans-unsaturation.

It is understood that the botanical materials can be varied to achieve desired benefits. For example, calendula may be added to the composition and is thought to stimulate tissue growth. Other exemplary botanical materials include, arnica for bruising, calendula for wound healing, chamomile for soothing. Some extracts may be added to improve the smell of the composition. Other botanical materials can be added based on their known properties, which are readily discovered in various research materials.

The inventor theorizes, without being bound to any particular theory, that at least one active agent or element from the botanical material is extracted into the carrier oil or the jojoba oil or esters. In some embodiments, the element is not readily identifiable but can be predictably prepared according to various methods herein. While there are numerous methods to prepare the therapeutic composition, in one specific embodiment, the therapeutic composition can be prepared by:

a) mixing a portion of a jojoba esters or oil with botanical materials selected from the group comprising: Plantago Lanceolata, Plantago major or combination thereof;

b) heating the mixture for a determined period of time, and in some cases the esters can melted and can be steeped for about 6 hours on temperature sufficient to keep the esters liquefied;

c) cooling the mixture or allowing the mixture to cool;

d) pulverizing the botanical materials (e.g. within the mixture);

e) optionally, steeping the mixture for a longer period of time (e.g. about 1 week to about 1 month or more); and

f) removing physically the botanical materials from the mixture.

Optionally, the method can include the step of formulating the mixture into a deliverable form (e.g. a gel or balm or cream). It is also possible to repeat any of the steps, including steps a) and b), to improve the yield of the elements into the esters or oil.

The ratio of Jojoba ester or oil to botanical materials (e.g. Plantago) in the composition can range from about 2.0 percent to about 50.0 percent. In one embodiment, the ratio of Jojoba ester to botanical material was about 4.25. In other examples, the ratio of Jojoba ester to botanical material ranged from about 5 percent to about 25 percent. In some examples, the portion of the Jojoba ester and the botanicals ranged from about 2 to about 20 percent of the composition. One of ordinary skill in the art can select the proper ratio of Jojoba ester or oil to botanical material based on the scale of the extraction. While Jojoba oil was used as an exemplary carrier oil, it is understood that the carrier oil can be any commercially acceptable oil, which includes oils from plants. For example, vegetable oils, i.e., castor oil, cottonseed oil, corn oil, can be used. Mineral oil or other synthetic oils can be used.

The time required for the extraction of the botanical material into the carrier oil or the jojoba oil can depend on the scale of the reaction. For 150 ounce reaction mixtures, the heating period may range from between 4 and 24 hours. The extraction can occur during times greater than and less than 24 hours, and the optimal time for a desired result can be determined without undue experimentation. It will be recognized by a person of ordinary skill in the art that extraction time can depends upon and varies with an array of conditions.

In order to improve the extraction of the botanical materials into the carrier oil or jojoba oil, the carrier oil can be heated (e.g., greater than 80 degree Celsius) and cooled; the botanicals can be pulverized; and/or the botanicals can steeped in the carrier oil. In one example, the botanical materials were steeped for about 1 month at 80 degrees. After which, the oil can be drained off or mixed with another oil (e.g. up to 5%) such as French Lavender essential oil. It is believed that about 1% or less of the at least one ingredient is extracted into the jojoba oil or carrier oil.

“Essential oils” include oils derived from herbs, flowers, trees, and other plants. Such oils can be extracted by several methods known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160 degrees to 240 degrees Celsius.

Essential oils typically are named by the plant from which the oil is found. For example, rose oil or peppermint oil are derived from rose or peppermint plants, respectively. Non-limiting examples of essential oils that can be used in the context of the present invention include tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

In one specific embodiment, the composition can include a wax such as Lanolin. Chemically, lanolin is a wax that is a mixture of esters of fatty acids with high molecular weight alcohols. For example, a typical high purity grade of lanolin is composed predominantly of long chain waxy esters (ca. 97% by weight) the remainder being lanolin alcohols, lanolin acids and lanolin hydrocarbons. There are more than 180 different acids and 80 alcohols identified. The melting point is about 40 degree Celsius. It is insoluble in water but forms very stable emulsions with it. It is known that lanolin can function as a medical material that can imitate and augment many of the functions of human skin lipids. In one example, a hypoallergenic Lanolin was used to extract the constituents from botanical materials, e.g. Plantago major. The lanolin can be about 10 to about 30% of the composition.

Further, one or more antimicrobial agent or antioxidant may be used to preserve and/or stabilize these compositions during manufacture and storage. For example, one or more of the large number of pharmaceutically acceptable preservatives can be used with various embodiments of this invention. Such preservatives include, but are not limited to, ethanol, ascorbyl palmitate, ethyl and methylparaben, butylated anisole, butylated hydroxytoluene, chlorobutanol, ethylenediamine, ethyl vanillin, monothioglycerol, phenethyl alcohol, benzoic acid and its salts. In one example, the composition was preserved with vitamin E oil. One of ordinary skill in the art can select one or more preservatives without undue experimentation.

The therapeutic composition can be formulated into a variety of liquids, solids and gel-like materials for delivery. The composition can be prepared as water and oil emulsions, anhydrous formulations. For example, these forms include liquids such as ointments, creams, gels, pastes and other forms. The composition may further include antioxidants, aromas/fragrances, vitamins, emulsifiers, toners, serums, lotions, elixirs, sun screens, etc. Antimicrobial, bactericidal, and thickening agents may also be included in a composition. In one specific embodiment, the composition is formed as an ointment.

In specific embodiments, silicone containing compounds and silicone wax compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids. The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less. The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively.

In one example, dimethicone could be used to prepare a gel-like material. Dimethicone is a commercially available silicone agent used as an ointment base. It could be present in the range from about 0.5 to about 95% by volume. In some examples, the dimethicone was ranged from about 2.5 to about 88.5% of the composition. In some embodiments, dimethicone can be used with lanolin. One of ordinary skill in the art can formulate the therapeutic composition as a gel without undue experimentation.

In another embodiment, the therapeutic composition can be combined with a sulfur (e.g. volcanic ash or silt), clay (e.g. Kaolin clay) and other ingredients (e.g. zinc oxide, calendula infused glycerine and polysorbate). It was found that composition could heal and kill infections including fungal infections common in animals (e.g. horses).

In use and application, the skin subject to treatment with the therapeutic composition showed improved healing and accelerated healing. An increase in the rate of re-epithelialisation of treated wounds and an acceleration of wound healing may be most readily demonstrated by an increased rate of re-epithelialisation of a wound. Such an increase in the rate at which the epithelial covering (for example the epidermis) is repaired or regenerated indicates that the healing of the wound in question has been accelerated. Accelerated healing of wounds may also be distinguished from “filling” of wounds that may occur through the generation of granulation tissue. Accelerated healing of a wound may lead to a treated wound healing at a rate at least 5% faster than an untreated wound or at a rate at least 10% faster or at least 15% or 100% (or more) faster. It was also found that certain embodiments were able to reduce or eliminate fungal infections in animals.

In addition, treatments with the therapeutic composition can result in improved appearance. Such improvements include improvements in the appearance of inflammation, scar appearance, non-inflammatory acne, surgical scars, rough or coarse textured skin, blotchy skin conditions, and sun damaged skin. The compositions were substantially non-toxic to the animals in which or on which it is placed.

The therapeutic composition can be applied as a “topical medicament” in that it is applied at a site where it is intended to have its effect. Topical medicaments suitable for use in accordance with the present invention include, but are not limited to, ointments; creams; lotions; gels; sprays; wound dressings capable of releasing active agents to the body; and injectable solutions administered by local injections (e.g. intradermal injections). Routes of administration are those that allow the provision of a therapeutically effective amount of an active compound to a connective tissue (such as the dermis, in the case of skin wounds) surrounding or underlying a wound, the healing of which is to be accelerated.

The amount of therapeutic composition to be applied can be determined by the area to be treated and can be therapeutically effective amount. When the therapeutic composition is applied to skin or wounds thereto, sufficient material can be dispensed so as to cover the affected area. A tool or device having an applicator can be used to apply various embodiments of the compositions described herein. Such tools or devices can apply a firm pressure across the skin to help create a uniform coat of the composition. Such devices can be powered or manual. In the case of the skin injuries, it can be appropriate to apply the composition manually. Further, the composition as lip balm can be applied manually.

The composition of the present invention can also be used in many cosmetic products including, but not limited to, sunscreen products, sunless skin tanning products, hair products, finger nail products, moisturizing creams, skin benefit creams and lotions, softeners, day lotions, gels, ointments, foundations, night creams, lipsticks, cleansers, toners, masks, skin-whiteners/brighteners, or other known cosmetic products or applications. Additionally, the cosmetic products can be formulated as leave-on or rinse-off products. In certain aspects, the compositions of the present invention are stand-alone products.

The application of the composition onto cuts, wounds, burns and ulcers is beneficial both in the cure of an infection or in the protection of the skin from infection. In all such cases, the composition of the present invention is easy to use, being semi-solid when applied and becoming liquid instantly upon rubbing onto the skin.

Specific examples of the composition can be used in treatment of wounds Skin wounds which can be treated using the compositions of the present invention include burn wounds, sunburn, cuts, abrasions, acute and chronic wounds and the like. Treatment of burn, ulcers, acute and chronic wounds typically is directed to keeping the wound as clean as possible and making the patient as comfortable as possible. It has been recognized in this respect that keeping the wound moist is advantageous to patient comfort. While maintaining a moist environment will effect some cooling of the tissue, it would be advantageous to be able to decrease the intradermal temperature of a burn wound, which would help to alter the progression of the tissue damage due to heat within the tissues.

Kits are also contemplated as being used in certain aspects of the present invention. For instance, compositions of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### EXAMPLES

The following examples are intended to illustrate various specific embodiments of the invention.

### 1. Preparation of a Therapeutic Composition

The botanical materials (Plantago lanceolata and/or Plantago major) were dehydrated in a dark room at 72 degrees for about 2 weeks. The Jojoba esters or oil were mixed with the botanical material without water. The ratio of Jojoba esters or oil to botanical was about 4.25, i.e., 1 gallon of jojoba esters was mixed with about 30 oz of botanical materials. The mixture was heated to about 90 degrees Celsius for 20 minutes. The botanicals were then pulverized, left uncovered to further cool, and then steeped for about 1 week at warm temperature (about 80 degrees Celsius). After the botanical materials were steeped, the botanical materials were removed from the mixture and the mixture was combined with a dermatologically acceptable carrier.

In another example, the lanolin extraction can be prepared by heating the lanolin and botanical materials at elevated temp from about 80 to 90 degrees Celsius. The botanical materials were pulverized in a mixer and allowed for about 12 hours or more. The heating step allowed the separation of the botanical material into the lanolin. The ratio of Lanolin to botanical was about 30 oz dried plant material to 1 gallon of lanolin.

### 2. Exemplary Compositions

In each of the examples, the botanical materials were first extracted using jojoba oil or esters. After which, the oil or esters were combined with the other materials to formulate a topical skin composition

Composition I had the following formulation:

| Material | Percentage (%) |
|---|---|
| Lanolin (medical grade) | 52 |
| Plantago Major Extract | 24 |
| Simmodsia Chinesis Seed Oil (Jojoba oil) | 19.5 |
| Stearoxy Dimethicone | 2.5 |
| D-alpha Tocopherol | 0.5 |
| Calendula Officinalis (Flower Extract) | 0.5 |
| Lavendula Officinalis (Essential Oil) | 1 |

Composition II had the following formulation:

| Material | Percentage (%) |
|---|---|
| Silicone | 31-35 |
| Silt containing Sulfur | 15 |
| Kaolin Clay | 20 |
| Plantago Major Extract | 2-4% |
| Zinc Oxide | 20 |
| Calendula Officinalis (Flower Extract) | 2.5 |
| Lemon & Orange extract | 1 |
| PCA Dimethicone | 1 |
| Azadirachta Indica (Neem) seed oil | 0.25 |
| Melaleuca Alternifolia (tea tree) Leaf Oil | 0.25 |
| Essential Oils | 1.5 |
| Phenoxyethanol, Caprylyl Glycol | 1 |

Composition III had the following formulation:

| Material | Percentage (%) |
|---|---|
| Silicone | 75 |
| Simmodsia Chinensis Esters | 12.5 |
| Plantago Major | 12.5 |

Composition IV had the following formulation:

| Material | Percentage (%) |
|---|---|
| Silt with sulfur (7%) | 25 |
| Kaolin clay | 8 |
| Zinc Oxide | 5 |
| Plantago Major Extract | 8.25 |
| Camphor | 3.5 |
| Composition III | 45 |
| Lavendula Officinalis Essential Oil | .25 |
| Azadirachta Indica (Neem) Seed Oil | 0.125 |
| Melaleuca Alternifolia (Tea Tree) Leaf Oil | 0.125 |
| Aloe Barbadensis Leaf Extract | 0.5 |
| LEMON & ORANGE EXTRACT | .5 |
| Polysorbate 80 | 1.75 |
| Undecyloyl glycine & Caproyl Glycine | 1 |
| Phenoxyethanol, Caprylyl Glycol | 1 |

Composition V had the following formulation:

| Material | Percentage (%) |
|---|---|
| Lanolin (medical grade) | 51 |
| Plantago Major Extract | 18 |
| Jojoba Oil | 26.5 |
| Stearoxy Dimethicone | 2.5 |
| Natural Vitamin E | 0.5 |
| Calendula Officinalis | 0.25 |
| Lavendula Officinalis (Essential Oil) | 0.25 |
| Zinc oxide | 0.75 |

Composition VI had the following formulation:

| Material | Percentage (%) |
|---|---|
| SILT WITHOUT SULFUR | 2.5 |
| Silt with Sulfur (7%) | 2.5 |
| Kaolin Clay | 25 |
| Zinc Oxide | 20 |
| Plantago Major Extract | 3 |
| Composition V | 38 |
| Cadendula Officinalis flower Extract | 5 |
| PCA Dimethicone | 0.5 |
| Azadirachta Indica (Neem) Seed Oil | 0.25 |
| Melaleuca Alternifolia (Tea Tree) Leaf Oil | 0.25 |
| Lemon & Fruit Extract | 0.5 |
| Lavendula Officinalis Essential Oil | 0.5 |
| Undecyloyl glycine & Caproyl Glycine | 1 |
| Phenoxyethanol, Caprylyl Glycol | 1 |

5. Extraction into Jojoba Oil

Jojoba oil was socked with the botanical materials to absorb the active constituents. The mixture was then heated and cooled. The product in Jajoba oil (1 ml) was extracted with 4 ml of methanol, which was evaporated to dryness, reconstituted with 1 ml of methanol which was subjected to UPLC/UV and UPLC/MS for analysis. UPLC/UV and UPLC/MS was used to analyze all the extract. Acquity UPLC BEH Shield RP18 1.7 μm, 100×2.1 mm column was used at 40° C. Total run time was 20 minute. Table 1 shows mobile phase for UPLC.

TABLE 1

| Time | Flow Rate | % ACN (0.05% FA) | % Water (0.05% FA) | Curve |
|---|---|---|---|---|
| 0.00 | 0.25 | 10.0 | 90.0 | 6 |
| 15.00 | 0.25 | 100.0 | 0 | 6 |
| 20.00 | 0.25 | 100.0 | 0.0 | 6 |

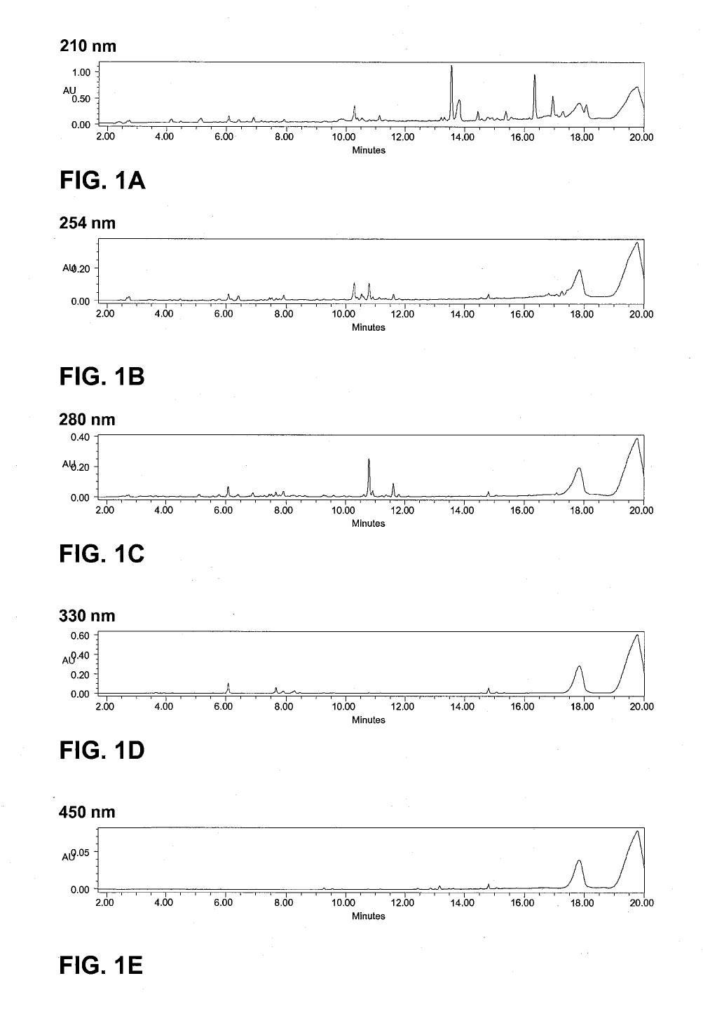

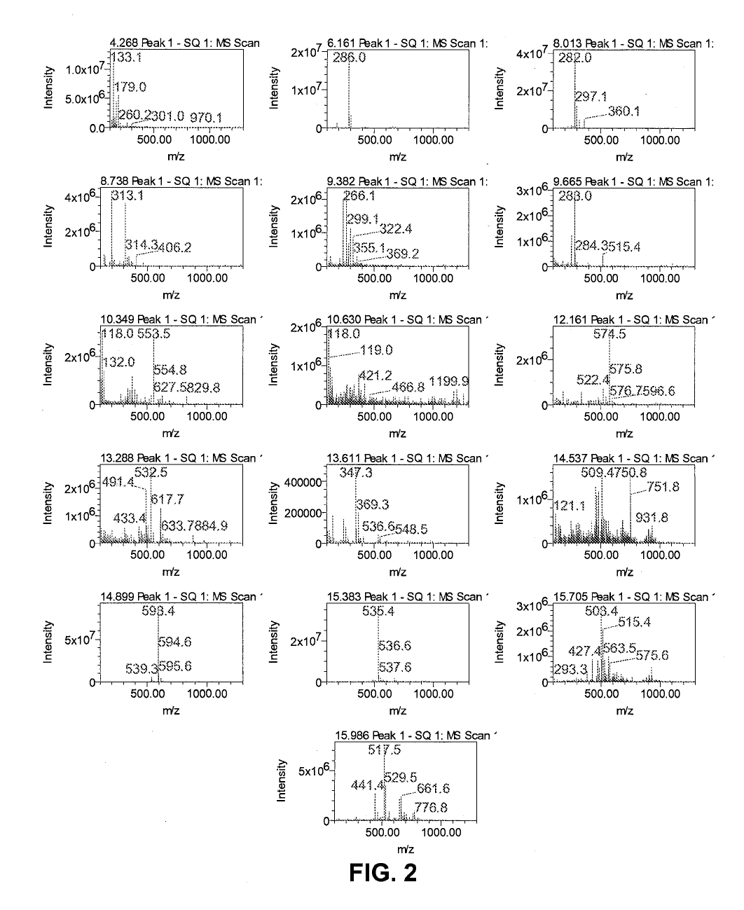

As can be seen in FIG. 1A through FIG. 1E, the oil contains contents that are identifiable from the UPLC chromatograms at different wavelength (210 nm, 254 nm, 280 nm, 330 nm and 450 nm. This finding is further supported by FIG. 2, which are the mass (ESI-positive ion mode) spectra of the different peaks of the extract of the jajoba oil, plantago extract.

### 6. A Multistep Healing Treatment

Jojoba oil was mixed with the botanicals Plantago lanceolata and/or Plantago major to absorb the active constituents contained therein. The botanicals were dehydrated in a dark room, at 72 degrees until all water has been depleted, which took about 2 weeks. The amount of botanical placed in the silicone was small as the silicone may not cover the plant material. The botanical material can be placed in increments to allow the leaf to soften (i.e. the heat) and then pulverize so that more plant material could be added to the oil. To get the concentration needed for this application, all the plant material was not be added at once. Ten ounces of Plantago lanceolata and/or Plantago major material were placed into a stainless pot and covered with about 5000 ml of dimethicone. The pot then was covered and allowed to sit overnight at room temperature.

The extraction process was complete, the plant material removed, and the Jojoba oil and the silicon were combined. The mixture was combined with carrier oil and then cleaned to remove plant material. The material was the therapeutic gel.

### 7. Examples Illustrating the Efficacy of the Compositions

The efficacy of the compositions or active ingredients within a given composition were determined by methods known to those of ordinary skill in the art. The following are non-limiting results that can be used in the context of various embodiments.

### Poison Ivy and Poison Sumac Dermatitis

In one case-study, a female patient, approximately 35 years of age, had contact dermatitis from poison sumac which was on the volar surfaces of both arms from the hand to the elbow. The patient had used various topical treatments including cortisone and Calamine without relief Composition I was applied to both arms and within about ten seconds, her itching was totally unnoticeable. She continued application of the Composition I, TID (three daily doses), and by four days, her skin had healed.

In another case-study, a male patient applied Composition I to poison ivy. He reported total alleviation of itching in less than 15 seconds.

### Insect Bites and Stings

In one case-study, a 55 year old female was stung on the hand by a wasp. Her reaction was typical with intense pain and immediate swelling. She applied Composition I to her hand and within one minute, the pain mitigated and by two minutes was totally unnoticeable. The swelling disappeared by 5 minutes and the site of the sting by 10 minutes was entirely healed.

In another case-study, a female approximately 60 years of age had a yellow-jacket sting her on both upper arms. The next morning, the pain continued and was substantial. The patient applied Composition I to both affected areas and within 10 seconds experienced total relief from the pain.

In another case-study, a 65 year old male was stung on the thumb by a scorpion. The wound bled profusely after being stung and the pain was described as intense. He applied Composition I and immediately the pain was relieved.

### Sunburn

In one case study, a 22 year old male had a moderate sunburn to which he applied Composition I. While the tightness in the skin was not relieved, the sunburn sting was totally relieved within 5 to 10 minutes. He continued to apply the product as needed, usually about every 4 hours.

### Eczema and Psoriasis

In one study, at least 10 patients have reported relief of symptoms from eczema after applying Composition I or Composition III to the affected areas of the skin. The itch was relieved within minutes and the scaly redness healed often within a couple of days.

One patient applied Composition I to a psoriasis lesion at the hairline on the back of neck. She reported that the area had intensely itched her for an entire day but after applying Composition I, the itch immediately was relieved. The epithelium, however, did not clear although she continued to experience total relief from the itching.

### Stage II Decubtis

In one study, the composition was shown to be effective against Stage II decubtis. An elderly, female, bed-ridden in a nursing home facility had a Stage II decubitis about 4×5 cm in the sacral area for over two years. Numerous products had been used prior with limited to no success. After application with various compositions, including Composition III, the decubitis closed in less than 14 days.

A representative elderly man who had a Stage II decubitis for 3 years had been treated previously (without limited success) with a variety of products and dressings. Healthcare professionals had also tried other methods but the decubitis failed to heal. Composition III was applied bid to the area and within eleven days, the wound was closed.

An representative elderly female who had extensive epithelial tears on her forearms was treated with Composition II. Within days, the epithelium healed completely.

Another representative male complained of widespread incontinent dermatitis that was producing severe burning. After applying Composition I, within 2 minutes the patient was totally relieved of pain and the rash resolved within 24 hours.

### Pediatric Diaper Dermatitis

About 40 babies, who have presented with various degrees of diaper dermatitis, from mild to excoriated blistered epithelium, have been successfully treated with Composition IV. It is common for the pain to be relieved with seconds and the rash to resolve in less than 24 hours.

### Radiation and Medical Oncology

Numerous patients have used Composition I both in a preventative fashion and for treatment for radiation dermatitis. Often, if the patient applies Composition I at simulation, radiation dermatitis is prevented or significantly delayed. In a palliative way, pain and burning are immediately relieved on contact after applying Composition I.

Patients who suffer Hand/Foot Syndrome as a sequel to the use of Xeloda® (Capecitabine) for metastatic breast or colon cancer have successfully used Composition I for treatment of their cracked hands and feet. Pain is relieved within minutes and epithelial repair usually occurs in less than 10 days.

The above detailed description of the embodiments, and the examples, are for illustrative purposes only and are not intended to limit the scope and spirit of the invention, and its equivalents, as defined by the appended claims. One skilled in the art will recognize that many variations can be made to the invention disclosed in this specification without departing from the scope and spirit of the invention.

What is claimed is:

1. The composition comprising at least one ingredient extracted into jojoba esters or jojoba oil from a botanical material selected from the group consisting of Plantago lanceolata, Plantago major, and a combination thereof.

2. The composition as claimed in claim 1, wherein the jojoba esters comprise long chain fatty acids and fatty alcohols joined by an ester bond.

3. The composition as claimed in claim 1, further comprising zinc oxide.

4. The composition as claimed in claim 1, further comprising clay.

5. The composition as claimed in claim 1, comprising a pharmaceutically acceptable additive.

6. The composition as claimed in claim 1, wherein the composition is substantially anhydrous.

7. The composition as claimed in claim 1, further comprising sulfur or a sulfur containing material.

8. The composition as claimed in claim 1, further comprising a fruit extract selected from the group consisting of orange, lemon, and combination thereof.

9. The composition as claimed in claim 1, further comprising an essential oil selected from the group consisting of seed oils.

10. A composition prepared by the steps comprising:
a) preparing a mixture of (i) a jojoba esters or oils, and (ii) a botanical material selected from the group consisting of Plantago lanceolata, Plantago major, and a combination thereof;
b) heating the mixture for a first predetermined heating period at a temperature;
c) cooling the mixture for a second determined period; and
d) isolating the at least one ingredient by physically removing the botanical material from the mixture.

11. The composition prepared using the steps claimed in claim 10, further comprising the step of steeping the mixture so to improve extraction of the at least one ingredient from the botanical material.

12. The composition further prepared by formulating the mixture into a deliverable form.

13. The composition prepared according to claim 10, further comprising the steps of heating the mixture for multiple periods of time and cooling the mixture for multiple periods of times.

14. The composition prepared according to claim 10, further comprising the step of: combining essential oil with the mixture.

15. The composition prepared according to claim 10, further comprising the step of: combining clay with the mixture.

16. The composition prepared according to claim 10, further comprising the step of pulverizing the botanical material within the mixture.

17. The composition prepared according to claim 10, wherein the aging step includes the step of storing the mixture in the dark for at least a predetermined time period.

18. The composition prepared according to claim 10, wherein the mixture is formulated into a topical application.

19. A method for treating skin comprising applying a composition as claimed in claim 1 to skin.

20. The method as claimed in claim 18, wherein said composition contains a therapeutically effective amount of botanical extracts for treating skin wounds.

21. A method for treating diseases of the skin comprising applying a therapeutically effective amount of the composition in claim 1 to skin.

22. A topical skin composition comprising:
(a) a combination of the following extracts selected from the group consisting of (i) at least one ingredient extracted into jojoba esters from a botanical material selected from the group consisting of Plantago lanceolata, Plantago major, and combination thereof, (ii) seed oil; and (iii) essential oil; and
(b) dermatologically acceptable carrier.

23. The topical skin composition of claim 22, wherein the dermatologically acceptable carrier comprises: (i) lanolin; and (ii) a preservative.

24. The topical skin composition, the composition is substantially anhydrous.

25. The topical skin composition of claim 22, wherein the dermatologically acceptable carrier comprises (i) 1 to 51% by weight of silicone; and (ii) 1 to 3% by weight of dimethicone.

26. A method for preparing a composition comprising:
(a) preparing a mixture of (i) a jojoba esters or oils, and (ii) a botanical material selected from the group consisting of Plantago lanceolata, Plantago major, and combination thereof;
(b) heating the mixture for a first predetermined heating period at a temperature;
(c) cooling the mixture for a second determined period; and
(d) isolating the at least one ingredient by physically removing the botanical material from the mixture.

27. The method as claimed in claim 26, wherein said composition contains a therapeutically effective amount of botanical extracts for treating skin wounds.
